SEQUENCE LISTING (3 sequences)

SEQ ID NO: 1 - DNA, 21 bp, synthetic construct
  source 1..21: mol_type = other DNA
gcatgaacagttgcccatact

SEQ ID NO: 2 - DNA, 21 bp, synthetic construct
  source 1..21: mol_type = other DNA
gagggagtagccatccgcatc

SEQ ID NO: 3 - protein, 1686 aa, synthetic construct
MDYKDHDGDYKDHDIDYKDDDDKMAPKKKRKVGIHGVPAADKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDKRPAATKKAGQAKKKKEFGSGEGRGSLLTCGDVEENPGPVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKEF

(19) United States
(12) Patent Application Publication
(10) Pub. No.: US 2024/0352129 A1
(43) Pub. Date: Oct. 24, 2024

(54) PROMOTING TRANSPLANTATION TOLERANCE

(71) Applicant: The Brigham and Women's Hospital, Inc., Boston, MA (US)

(72) Inventors: Jamil Azzi, Chestnut Hill, MA (US); Nour Younis, Cambridge, MA (US); Omar Abudayyeh, Cambridge, MA (US)

(21) Appl. No.: 18/628,258

(22) Filed: Apr. 5, 2024

Related U.S. Application Data
(60) Provisional application No. 63/457,994, filed on Apr. 7, 2023.

(51) Int. Cl. C07K 16/28; A61K 39/00; A61P 37/06; C12N 5/0783; C12N 9/22; C12N 15/11; C12N 15/90

(52) U.S. Cl. CPC C07K 16/2833; A61K 39/4611; A61P 37/06; C12N 5/0636; C12N 9/22; C12N 15/11; C12N 15/907; C07K 2317/24; C12N 2310/20; C12N 2510/00

(57) ABSTRACT

Disclosed are compositions and methods of promoting transplantation tolerance, treating an immune disorder associated with graft transplantation, and promoting organ survival in an organ transplanted into a subject. The methods include administering anti-CD74 antibodies or CD74-deficient Treg cells.

### CLAIM OF PRIORITY

This application claims priority under 35 USC § 119 (e) to U.S. Patent Application Ser. No. 63/457,994, filed on Apr. 7, 2023. The entire contents of the foregoing are hereby incorporated by reference.

### SEQUENCE LISTING

This application contains a Sequence Listing that has been submitted electronically as an XML file named “29618-0437001 SL_ST26.XML.” The XML file, created on Jun. 4, 2024, is 5,359 bytes in size. The material in the XML file is hereby incorporated by reference in its entirety.

### STATEMENT REGARDING FEDERALLY FUNDED RESEARCH

This invention was made with government support under Grant No. AI179986 awarded by The National Institutes of Health. The government has certain rights in the invention.

### BACKGROUND OF THE INVENTION

Organ transplantation to treat end-stage organ disease has improved over the years, but chronic rejection and immunosuppressant side effects remain a significant concern. Regulatory T cells (Tregs) play an essential role in maintaining tolerance to self-antigens, preventing excessive immune responses and abrogating autoimmunity. Due to their suppressive properties, Tregs have been extensively studied for their use as a cellular therapy to treat Graft versus Host Disease and limit immune responses responsible for graft rejection. Inflammation is known to negatively affect Tregs stability and suppressive function, impair the induction of solid organ transplant tolerance, and enhances acute and chronic rejection, leading to worse long-term outcomes for transplant recipients [1-3].

### SUMMARY OF THE INVENTION

Insights into key inflammatory pathways that suppress Tregs during alloimmune activation can revolutionize our therapeutic approach to promoting stable, endogenous Tregs and improving long-term outcomes in organ transplantation. This effect can be substantially potentiated if the molecular target exerts a differential effect on Tregs versus alloreactive cells, resulting in superior Tregs function and impaired pro-inflammatory activity of alloreactive cells.

A serendipitous discovery has identified such a critical inflammatory pathway. We observed an increase in CD74 gene expression in urinary extracellular vesicles of rejecting kidney transplant recipients, which led to surprising observations [4]. We show for the first time in fully-mismatched murine heart transplantation that graft infiltrating Teff cells, but not Treg cells, secrete an inflammatory cytokine, macrophage migration inhibitory factor (MIF) and that both graft infiltrating Treg cells and Teff cells upregulate on their cell surface the MIF receptor called CD74. MIF works in an autocrine fashion in Teff cells to promote their function and in a paracrine fashion in Treg cells to suppress immune regulation. Interestingly, genetic ablation of CD74 in mice leads to indefinite survival of allogeneic grafts by significantly promoting Treg cell expansion within the allografts and by reprogramming Teff cells toward exhaustion.

Accordingly, in one aspect the invention is a method of promoting transplantation tolerance in a subject by administering a therapeutically effective amount of an anti-CD-74 antibody to the subject. The anti-CD74 antibody can be administered prior to, simultaneously with, or subsequent to transplantation of the organ into the subject. The anti-CD74 antibody, Milatuzumab (Invitrogen MA5-41757), can be used for example. The subject is preferably a human subject but could be any other mammal undergoing organ transplantation.

In another aspect, the invention is a method of treating an immune disorder associated with graft transplantation in a subject by administering a therapeutically effective amount of an anti-CD-74 antibody to the subject. The anti-CD74 antibody can be administered prior to, simultaneously with, or subsequent to transplantation of the organ into the subject. The anti-CD74 antibody, Milatuzumab (Invitrogen MA5-41757), can be used for example. The subject is preferably a human subject but could be any other mammal undergoing organ transplantation.

In yet another aspect the invention is a method of promoting organ survival in an organ transplanted into a subject by administering a therapeutically effective amount of an anti-CD-74 antibody to the subject. The anti-CD74 antibody can be administered prior to, simultaneously with, or subsequent to transplantation of the organ into the subject. The anti-CD74 antibody can be Milatuzumab (Invitrogen MA5-41757) can be used for example. The subject is preferably a human subject but could be any other mammal undergoing organ transplantation.

In another aspect the invention is a composition including CD74 deficient Treg cells. The CD74 deficient Treg cells are characterized as being more immunosuppressive than wild type Tregs. They also release higher levels of suppressive cytokines, for example Granzyme B, and they express higher levels of co-inhibitory molecules, for example PD1. The C+CD74 deficient Treg cells can be made by, for example, by using a CRISPR/Cas genome editing process, such as the process disclosed herein.

In yet another aspect, the invention is a method of promoting transplantation tolerance in a subject by administering a therapeutically effective amount of a composition of CD74 deficient T reg cells to the subject. The composition can be administered prior to, simultaneously with, or subsequent to transplantation of the organ into the subject. The subject is preferably a human subject but could be any other mammal undergoing organ transplantation.

In another aspect, the invention is a method of treating an immune disorder associated with graft transplantation in a subject by administering a therapeutically effective amount of a composition of CD74 deficient T reg cells to the subject. The composition can be administered prior to, simultaneously with, or subsequent to transplantation of the organ into the subject. The subject is preferably a human subject but could be any other mammal undergoing organ transplantation.

In another aspect, a method of promoting organ survival in an organ transplanted into a subject by administering a therapeutically effective amount of a composition of CD74 deficient T reg cells to the subject. The composition can be administered prior to, simultaneously with, or subsequent to transplantation of the organ into the subject. The subject is preferably a human subject but could be any other mammal undergoing organ transplantation.

Additionally, provided herein are methods of preparing a population of CD74-deficient T reg cells. The methods comprise providing a population of CD4+Foxp3+Treg cells, or precursors thereof; and using a Cas enzyme to edit genomic DNA of the cells to introduce a mutation that knocks out CD74; thereby preparing a population of CD74-deficient T reg cells. The CD74 deficient Tregs can be sorted, e.g., flow-sorted, based on expression of a fluorescent reporter protein, e.g., GFP. In some embodiments, the precursors are CD34+ hematopoietic stem cells (HSCs) or induced pluripotent stem cells (iPSCs), and the methods further comprise maturing the precursors in vitro to produce Treg cells. In some embodiments, the Cas enzyme is a nuclease or nickase (e.g., Cas9, e.g., SpCas9 or saCas9 or an optimized version thereof. In some embodiments, the Cas enzyme lacks nuclease activity, e.g., is in a base editor or prime editor. In some embodiments, the Cas enzyme is Streptococcus pyogenes Cas9 (SpCas9) and the gRNAs comprise spacer sequences CD74_G1: GCATGAACAGTTGCCCATACT (SEQ ID NO: 1), CD74_G2: GAGGGAGTAGCCATCCGCATC (SEQ ID NO:2). In some embodiments, the cells are from a human subject.

Also provided herein are compositions comprising CD74 deficient Treg cells, e.g., prepared by a method described herein. In some embodiments, the CD74-deficient Treg cells exhibit more immunosuppression as compared to wild type Treg cells. In some embodiments, the CD74 deficient Treg cells release higher levels of suppressive cytokines as compared to wild type Treg cells. In some embodiments, the CD74 deficient Treg cells express higher levels of co-inhibitory molecules as compared to wild type Treg cells.

Further, provided herein are methods for promoting transplantation tolerance in a subject, or promoting organ survival in an organ transplanted into a subject. The methods comprise administering a therapeutically effective amount of CD74 deficient Treg cells as described herein to the subject. In some embodiments, the CD74 deficient Treg cells are administered prior to, simultaneously to, or subsequent to, transplantation of an organ into the subject. In some embodiments, the subject is a human subject. In some embodiments, the CD74 deficient Treg cells are prepared using CD4+Foxp3+Treg cells, or precursors thereof that are autologous to the subject.

The composition used in the foregoing methods can be prepared ex vivo by the CRISPR/Cas process, as is known in the art. In all of the above methods, a therapeutically effective amount means an amount of a compound that, when administered to a mammal, e.g., a human, for treating a disease or condition, is sufficient to effect such treatment for the disease or condition. The “therapeutically effective amount” will vary depending on the compound, the disease and its severity and the age, weight, etc., of the mammal to be treated as is well known in the art.

The term “subject” refers to any animal (e.g., a mammal), including, but not limited to, humans and non-human veterinary subjects including non-human primates, and is used interchangeably with the term “patient”.

As used herein, reference to “about” or “approximately” a value or parameter includes (and describes) embodiments that are directed to that value or parameter. For example, a description referring to “about X” includes description of “X”. “About” as used herein means plus or minus 10 percent.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF THE DRAWINGS

FIGS. 1A-I: A. In our previous work on mRNA isolated from the urinary extracellular vesicles of transplant patients, CD74 was among the highest gene enriched during rejection. B. CD74KO and CD74 fl/fl were generated using CRISPR-Cas9 technology. C. Kaplan-Meier allograft survival curve of BALB/c donor heart in WT vs CD74KO recipient mice (n=6 in each group). D. Histological analysis of rejection hallmarks in WT vs CD74KO allograft. CD74KO allograft showed significantly less fibrosis and cellular infiltrate. E. Representative flow cytometry plots showing up to 3-fold higher Treg infiltrate in the CD74KO allograft, compared to WT allograft. F. Graph displaying % of Tregs and % of PD1⁺ Tregs in allografts, draining Lymph nodes (dLN), and spleens (SPL) of WT vs CD74KO recipients. G. WT vs CD74KO allograft stained with intracellular Foxp3, red dots represent infiltrating Tregs. H. Frequency of CD74 expression on Tregs in heart allograft, draining LN and spleen harvested from WT recipients of BALB/c heart on day 7 post-transplantation. I. Kaplan-Meier allograft survival curve of BALB/c donor heart in WT vs CD74KO recipient mice with or without Treg depletion, n=6 for control and 3 for anti-CD25 treated. One-way ANOVA with Tukey's Multiple Comparison Test was used to evaluate differences between groups while student t-test was used for pairwise comparisons. Differences between groups were considered significant at p-values below 0.05 (*p<0.05, **p<0.01, ***p<0.001, ****p<0.0001).

FIGS. 2A-B. A. Mean fluorescent intensity of CD74 in CD4 effector cells, CD8 effector cells, Tregs, and ex-Tregs (Tregs that lost their Foxp3 expression after stimulation). B. Mean fluorescent intensity of MIF in CD4 effector cells, CD8 effector cells, Tregs, and ex-Tregs. One-way ANOVA with Tukey's Multiple Comparison Test was used to evaluate differences between groups. Differences between groups were considered significant at p-values below 0.05 (*p<0.05, **p<0.01, ***p<0.001, ****p<0.0001).

FIGS. 3A-D. A. Suppression assay using CD74KO vs WT Tregs on activated CD4⁺CD25⁻ T cells, higher proliferation reflected by lower CTV concentration. B. Suppression assay using WT Tregs on activated CD4⁺CD25⁻ T cells, higher proliferation reflected by lower CTV concentration. Blocking CD74 with an anti-CD74 monoclonal antibody resulted in higher suppression. Graph showing that anti-CD74 had no effect on non-Tregs proliferation when stimulated in the absence of Tregs. C. Treg proliferation in vitro, assessed by CTV higher dilution with increased proliferation, CD74KO Tregs (blue) and WT Tregs (Red). Proliferation index reflects cell proliferation. Treg viability, reflected by % of Live cells, was similar between groups. Frequency of FoxP3⁺ cells, after 5 days of stimulation in culture and having started with same purity, was significantly higher in CD74KO Tregs. D. Frequency of PD-1⁺ and GrB⁺ in activated Tregs. All experiments were repeated three separate times. Student t-test was used for pairwise comparisons. Differences between groups were considered significant at p-values below 0.05 (*p<0.05, **p<0.01, ***p<0.001, ****p<0.0001).

FIGS. 4A-E. A. UMAP showing distinctive CD4⁺ populations, including suppressive Tregs, central Tregs, and effector CD4 cells. B. Volcano plot showing the top differentially expressed genes between CD74KO (Blue) and WT (Red). C. Transcription factor analysis using differentially expressed genes showing the upregulation of IRF1 signaling in wild type compared to CD74KO (upper graph); Wild type, when compared to CD74KO, showed a similar phenotype as Foxp3 deficient mice (lower graph). D. KLRG1 and Helios expression on WT vs. CD74KO Tregs infiltrating the allograft. E. MIF stimulation of Tregs upregulated IRF1 in WT but not in CD74KO.

FIGS. 5A-C. A. CD74KO recipients of BALB/c hearts had significantly higher % of CD8⁺ and CD8⁺CD44highCD62Llow cells (CD8 effector cells) in their dLNs, compared to WT mice. However, these cells expressed higher LAG3 and PD1 and lower CD127 and released significantly less GrB. B. CD74KO CD8 effector cells exhibited significantly lower memory response to donor antigen in a mixed lymphocytic reaction as evident by both % of Proliferating Cells (tracked by CTV) and % of Ki67⁺ cells. They also released significantly less IFN-γ than WT cells. C. CD74KO recipients of BALB/c hearts had significantly lower % of CD4⁺ in their dLNs, compared to WT mice. However, they had a higher % of CD4 effector cells out of CD4 cells. CD74KO CD4 effector cells displayed a similar exhaustion phenotype as CD8 effector cells (they express higher LAG3 and PD1, and lower CD127, compared to WT CD4 effector cells). In vivo experiments were repeated three times. Student t-test was used for pairwise comparisons. Differences between groups were considered significant at p-values below 0.05 (*p<0.05, **p<0.01, ***p<0.001, ****p<0.0001).

FIGS. 6A-C. A. CD4 T cells proliferation in vitro, assessed by CTV, and % of Proliferating WT vs. CD74KO T effector cells (p-value: 0.006). B. CD8 T cells proliferation in vitro, assessed by CTV, and % of Proliferating WT vs. CD74KO T effector cells (p-value: 0.009). C. Kaplan-Meier allograft survival curve of BALB/c donor heart in RAGKO recipient mice injected with WT or CD74KO CD3⁺CD25⁻ cells.

FIGS. 7A-B. A. Trajectory analysis showing more spilling of suppressive Tregs cluster into the CD4 effector cells cluster in WT, but not in CD74KO. B. TCR clonal analysis suggesting that allograft-infiltrating CD74KO Tregs are less clonally diverse than WT Tregs. All experiments were repeated three separate times. Student t-test was used for pairwise comparisons. Differences between groups were considered significant at p-values below 0.05 (*p<0.05, **p<0.01, ***p<0.001, ****p<0.0001).

FIGS. 8A-E. A. Histogram showing IRF1 expression in response to IFNg in WT vs CD74KO Tregs. CD74KO Tregs were less responsive to IFNg as evident by their weak upregulation of IRF1 in response to IFNg. B. Graph showing % of IFNgR1 expression by WT vs CD74KO allograft-infiltrating Tregs. C. Graph showing the effect of IFNg on Tregs stability and IRF1 expression. D. CD74 (brown) and IFNg (yellow) binding potential-confidence score 91.5% and docking score −268. E. CD74 (brown) and IFNg signaling complex (yellow) binding potential-confidence score 98% and docking score −348.

FIGS. 9A-D. A. Kaplan-Meier allograft survival curve of BALB/c donor heart in WT vs CD74fl/fl-UBC cre recipient mice. B. Graph displaying % of Tregs in the allografts of WT and CD74fl/fl-UBC cre recipients. C-D. Graph and Representative flow cytometry plots showing PD1, KLRG1, Helios, and GrB expression by WT vs CD74fl/fl-UBC cre Tregs.

### DETAILED DESCRIPTION

### Macrophage Migration Inhibitory Factor (MIF) and CD74:

MIF is a potent pro-inflammatory cytokine produced and released by immune cells, particularly macrophages, in response to bacterial toxins and physiologic stressors [6]. MIF plays an integral role in innate immunity. It potentiates the anti-microbial activity of macrophages by releasing pro-inflammatory cytokines [6]. Additionally, MIF is a central contributor to systemic inflammatory disorders like sepsis [7-9]. However, its role in adaptive immunity is not well known.

CD74 is a transmembrane receptor [10, 11]. MIF exerts its effects upon binding to the extracellular domain of CD74, resulting in a cascade of intracellular signaling events that favors inflammation and autoimmunity [12-14]. Upon binding to MIF, CD74 can affect cellular proliferation, migration, and survival via its intracellular domain (ICD) that cleaves and acts as a transcription factor [10, 11, 15]. CD74 has also been described as the invariant chain of the MHC-II molecule and acts as a chaperone for this molecule [10, 11]. However, MHC-II antigen processing is critical in immune tolerance and deleting MHC class II abrogated tolerance in murine transplant model contrary to mice deficient in CD74 [16, 17]. Furthermore, our data show an intrinsic role for CD74 in Tregs and Teff.

The data presented in the examples below reveals that CD74 deficient Tregs are more suppressive than wild type Tregs. They also release higher levels of suppressive cytokines (i.e. Granzyme B), and express higher levels of co-inhibitory molecules (i.e. PD1), which are vital for Tregs suppressive function. Additionally, CD74 deficient Tregs are more stable than wild type Tregs; they exhibit higher proliferation in-vitro and maintain higher Foxp3 expression after 5 days of robust stimulation. Together, these findings suggest that CD74 deletion in Tregs can suppress the unchecked pro-inflammatory activity of cytotoxic cells promoting both auto- and allo-tolerance (tolerance to self and non-self). CD74 deficient human Tregs can be found useful in the field of Tregs therapy, as they can be employed to create superior antigen specific Tregs or CAR-Tregs for allotransplantation. Thus, provided herein are methods of reducing CD74 expression in human Tregs, either in vivo by administration of an anti-CD74 antibody, or ex vivo using genome editing. The edited Tregs or anti-CD74 antibody can then be administered to subjects undergoing organ transplantation.

### Methods of Treatment

The anti-CD74 antibodies or CD74-deficient Treg cells described herein can be used for treating or preventing (i.e., reducing risk of) transplant rejection in a subject in need thereof. As described herein, anti-CD74 antibodies or CD74-deficient Treg cells are therapeutic in these diseases. In some embodiments, the transplant rejection can be a rejection of a kidney, lung, heart, liver, limb, skin, or multi-organ transplant. In some embodiments, the subject does not have cancer, e.g., does not have a blood cancer, e.g., does not have lymphoma, leukemia, or multiple myeloma. In some embodiments, the subject is a transplant subject who has levels of CD74 in urinary exosomes above a reference level that represents a level in a subject who is not undergoing rejection or not undergoing CD74-mediated transplant rejection.

In various aspects and embodiments, the disclosure provides methods for treating or preventing transplant rejection. The transplant rejection can be, for example, kidney rejection.

“Administration” and “treatment,” as it applies to an animal, human, experimental subject, cell, tissue, organ, or biological fluid, can include contacting an exogenous pharmaceutical, therapeutic agent, diagnostic agent, or composition to the animal, human, subject, cell, tissue, organ, or biological fluid. “Administration” and “treatment” include in vivo, as well as in some embodiments in vitro or ex vivo treatments.

Typically, the anti-CD74 antibodies or CD74-deficient Treg cells is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom can vary according to factors such as the disease state, age, and weight of the subject, and the ability of the drug to elicit a desired response in the subject. Whether a disease symptom has been alleviated can be assessed by any clinical measurement typically used by physicians or other skilled healthcare providers to assess the severity or progression status of that symptom.

As such, in various embodiments, the term “effective amount” or therapeutically effective amount” is a concentration or amount of the anti-CD74 antibodies or CD74-deficient Treg cells that results in achieving a particular stated purpose, e.g., reduction in one or more symptoms of a disease described herein. An “effective amount” of anti-CD74 antibodies or CD74-deficient Treg cells can be determined empirically. Furthermore, a “therapeutically effective amount” is a concentration or amount of anti-CD74 antibodies or CD74-deficient Treg cells that is effective for achieving a stated therapeutic effect (e.g., reduction in symptoms or prevention/delay of transplant rejection). This amount can also be determined empirically.

### Anti-CD74 Antibodies

The present methods can include the use of anti-CD74 antibodies, a number of which are known in the art. Preferably the antibodies are human or humanized; exemplary antibodies include Milatuzumab (hLL1) (see, e.g., WO2003074567, other known and/or commercially available anti-CD74 antibodies may be utilized, such as At14/19; 1D1; 1B8; 2D1B11; 2D1B3; 332516; 3D7; 5-329; 6D9; B318; Bu45; BU45; CDLA74-1; CerCLIP.1; CLIP/1133; CLIP/813; LN2; LN-2; M-B741; N2; OTI1G10; OTI1H3; PIN. 1; PIN1; SPM523; UMAB231; or UMAB232 (commercially available from LSBio, Seattle, WA; Biolegend, San Diego, CA; Abcam, Cambridge, MA; Serotec, Raleigh, NC; Abnova, Taipei City, Taiwan; and ebioscience, San Diego, CA. The antibodies can be administered using methods known in the art, e.g., parenteral administration, optionally intravenous or subcutaneous administration, using a composition suitable for such administration.

### CD74-Deficient Treg Cells

The present methods can include using CD74-deficient Treg cells, and methods of making them using CRISPR/Cas editing. As used herein, CD74-deficient Treg cells have reduced levels of expression of CD74, e.g., reduced by at least 20%, or at least 30%, 40%, 50%, 60%, 70%, 80%, 90%, or about 100% as compared with unedited Treg. In some embodiments, CD74-deficient Treg cells have levels of expression of CD74 reduced by at least about 50% to about 100% as compared with unedited Treg.

The methods can include editing a Treg cell (e.g., a mature CD4+Foxp3+ Treg, or a precursor thereof (e.g., CD34+ hematopoietic stem cells (HSCs) or induced pluripotent stem cells (iPSCs) that are then matured in vitro to produce Treg cells. Preferably the cells are human cells. CRISPR/Cas editing can be used to induce edits that result in non-functional alleles of CD74, e.g., by base editing to insert a mutation resulting in a premature stop codon, or by inducing one or a pair of double stranded breaks resulting in deletion of one or more nucleotides to create a deletion, frame shift, or other mutation resulting in either loss of transcription, loss of translation, or production of a non-functional protein (see, e.g., Ran et al., Nat Protoc. 2013 November; 8 (11): 2281-2308). In general, the methods include introducing into the cell a CRISPR/Cas enzyme, e.g., Cas9, and a guide RNA that complexes with the Cas enzyme to direct its activity to a target sequence in the genome. The Cas enzyme can be introduced as a protein (e.g., in a ribonucleoprotein (RNP) complex with the guide RNA) or as a nucleic acid encoding the protein and/or guide RNA. Cas nucleases, nickases, and Cas-based base editors and prime editors, and optimized versions thereof are known in the art and can be used in Tregs. Several kits are commercially available for CRISPR/Cas editing to knockout CD74 expression, e.g., from Santa Cruz Biotech, Applied Biological Materials Inc., and OriGene Technologies, Inc. An exemplary sequence of human CD74 is available in GenBank at NC_000084.7 (Reference GRCm39 C57BL/6J), Range 60936921-60945724). See also Van Zeebroeck et al., Front Immunol. 2021 Aug. 2:12:655122; Kondrashov et al., Int J Mol Sci. 2022 February; 23 (3): 1689; Amini et al., Front Immunol. 2021 Feb. 24:11:611638; Amini et al., Front Immunol. 2020; 11:611638.

When intended for use in treating a subject, the Tregs or precursors thereof can be autologous to the subject (i.e., derived from the subject). In other embodiments, the Tregs or precursors are allogeneic to the subject (i.e., derived from a different subject). Also provided herein are CD74-deficient Tregs, e.g., made by a method described herein, and compositions comprising the CD74-deficient Tregs. The CD74-deficient Tregs can be administered using methods known in the art, e.g., parenteral administration, e.g., intravenous administration, using a composition suitable for such administration.

### EXAMPLES

The invention is further described in the following examples, which do not limit the scope of the invention described in the claims.

### Methods and Materials

The following methods and materials were used in the Examples herein.

### Mice

C57BL/6, BALB/c and B6.129S7-Rag1tm1Mom (Rag1KO) and UBC-Cre mice were purchased from the Jackson laboratories. CD74KO and CD74flox/flox (fl/fl) mice were obtained from the laboratory of Richard Buccala (Yale University, Connecticut, USA). CD74fl/fl-UBC-cre-ERT2 mice were bred in-house. All mouse strains used in this research were housed under specific pathogen-free conditions at Brigham and Women's Hospital animal facility, adhering to federal, state, and institutional protocols. The research protocol received approval from the Brigham and Women's Hospital Institutional Animal Care and Use Committee. Mice were matched for sex and age (8 to 12 weeks old).

### Heterotopic Heart Transplantation.

BALB/c hearts were transplanted into the abdominal cavity of recipient C57BL/6 (WT), CD74KO, CD74fl/fl-UBC-cre-ERT2 or RAGKO as previously described [30]. Mice were euthanized at day 7 post-transplantation for mechanistic analysis. For survival studies, allograft rejection was monitored daily and confirmed by the absence of palpable heartbeats. Clinical allograft rejection endpoint was set at day 100 post-transplantation.

### Immunophenotyping

WT and CD74KO hosts of BALB/c hearts were euthanized at day 7 post-transplantation. Cell suspensions from allografts, draining lymph nodes (para-aortic lymph nodes), and spleens were stained and analyzed using flow cytometry. Anti-CD4 PercpCy5.5 (RM4-4, #116012), anti-CD62L (MEL-14, #104420), anti-CD25 PE (3C7, #101904), anti-PD1 BV605 (29F.1A12, #135220), anti-Granzyme B PE-Cy7 (QA 16A02, #372214), anti- KLRG1 PE-Cy7 (2F1/KLRG1, #138415) were purchased from Biolegend. Anti-Foxp3 APC (FJK-16s, #17-5773-82) and anti-Helios PE-Cy5 (22F6, #15-9883-42) were purchased from Invitrogen. Anti-CD74 BV421 (In1, #740057), anti-CD8 BUV805 (53-6.7, #612898), anti-CD45 BUV395 (30-F11, #564279), and anti-CD44 BV510 (IM7, #563114) were purchased from BD Biosciences. Cytek Aurora (5 Laser 16UV-16V-14B-10YG-8R) was used for flow cytometry and the result was analyzed with FlowJo version 10 (Flowjo LLC).

### Tregs Suppression Assay

CD4+CD25+ and CD4+CD25− cells (T responder cells) were FACS-isolated from WT or CD74KO mice. 5×104 CD4+CD25− cells were labeled with Cell Trace Violet (Invitrogen, #C34557), and co-cultured with or without 2.5×104 CD4+CD25+ cells in 96-well flat bottom tissue cultured plate coated with 3 ug/ml anti-CD3 (Invitrogen, 45-2C11, #16-0031-85) and 3 ug/ml anti-CD28 (Invitrogen, 37.51, #16-0281-85) for 72 hours. The proliferation of CD4+CD25− cells was assessed using Cell Trace Violet labeling and analyzed using Flowjo v10 software. To study the effect of CD74 blocking on Tregs suppressive function, anti-CD74 monoclonal antibody was obtained from Richard Buccala Laboratory and added to activated WT T responder cells cultured with WT Tregs at a concentration of 125 ug/ml.

### Tregs Expansion

CD4+CD25+ were FACS-isolated from WT or CD74KO mice. 5×105 CD4+CD25+ cells were labeled with Cell Trace violet and activated with anti-CD3/CD28 dyna beads (Gibco 11456D) in the presence of IL-2 (10 ng/ml; Peprotech, #200-02) for 5 days. Cells proliferation was measured using Cell Trace Violet labeling analyzed using Flowjo v10 software. We assayed the percentage of CD25+Foxp3+ of CD4 cells using flow cytometry.

### Single-Cell RNA Sequencing Analysis

Hashtag oligos (HTOs) sparse matrix output from the CellRanger pipeline was loaded into R using the Seurat package. Doublets and negative cells were filtered out and only singlets were kept for analysis. The count matrix was normalized using “NormalizedData” function and 2000 variable features were used to identify the highly variable genes using “FindVariableFeatures” function. Any TCR genes were excluded from the highly variable genes to avoid obtaining clusters of TCR genes. Integration of the samples was done using the canonical correlation analysis (CCA) provided by Seurat. “SelectIntegrationFeatures”, “FindIntegrationAnchors” and “IntegrateData” were run sequentially to integrate the samples, followed by scaling and dimensionality reduction using “ScaleData”, “RunPCA” and “RunUMAP”, respectively. Clustering at different resolutions was conducted using “FindNeighbours” and “FindClusters” functions.

Marker genes were identified at the resolution of 0.4 using the “FindAllMarkers” Seurat function, with the following parameters “min.pct” of 0.1 and “logfc.threshold” of 0.25. Clusters' comparisons between conditions was performed using “FindMarkers” function. Gene set enrichment analysis was performed using GSEA package developed by Broad Institute. Clonotypes comparison between conditions and clonal diversity was done using the scRepertoire R/Bioconductor package.

### CD4 and CD8 Expansion

CD4+CD25− and CD8 cells were FACS-isolated from WT and CD74KO mice, and labeled with Cell Trace Violet. 5×104 Cells were plated in a 96-well flat bottom plate coated with anti-CD3 and anti-CD28 and cultured for 72 hours. Cell proliferation was assessed using Cell Trace Violet labeling and analyzed using Flowjo v10 software.

### Mixed Lymphocyte Reaction Assay

Naïve donor BALB/c splenocytes were irradiated and co-cultured as antigen-presenting cells with recipient WT or CD74KO CD8+CD44+ cells isolated from the draining lymph nodes of the euthanized animals at day 7 post transplantation. Cell Trace Violet labeling and Ki67 intracellular staining (PerCP/Cyanine5.5, 16A8, 652424) were used to assess recipient cells proliferation.

### Adoptive T Cell Transfer Models

RAGKO mice were transplanted with BALB/c hearts a week prior to adoptive cell transfer. WT or CD74KO CD3+CD25− cells (7.0×105) were FACS-sorted and injected into the RAGKO hosts. Allograft rejection was monitored daily until day 100 post adoptive transfer.

### Statistics

Statistical analyses were conducted using Prism 8.2.1 software (GraphPad Software). Statistically significant results were defined as P values less than 0.05 (*=<0.05, **=<0.01, ***=<0.001).

### Example 1. CD74 Deficiency Induces Indefinite Allograft Survival of Fully Mismatched Murine Cardiac Transplant

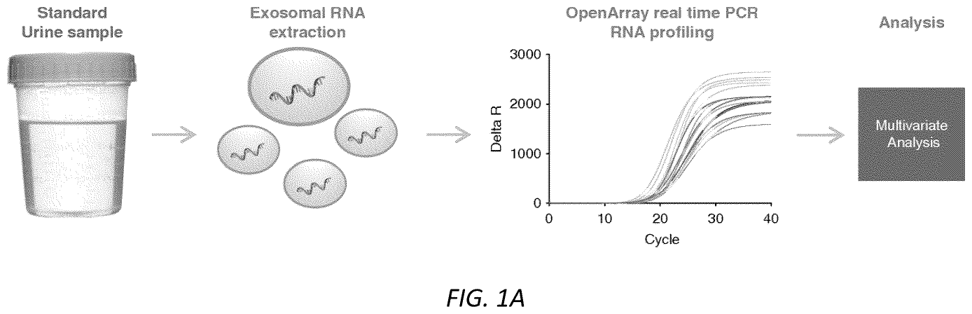

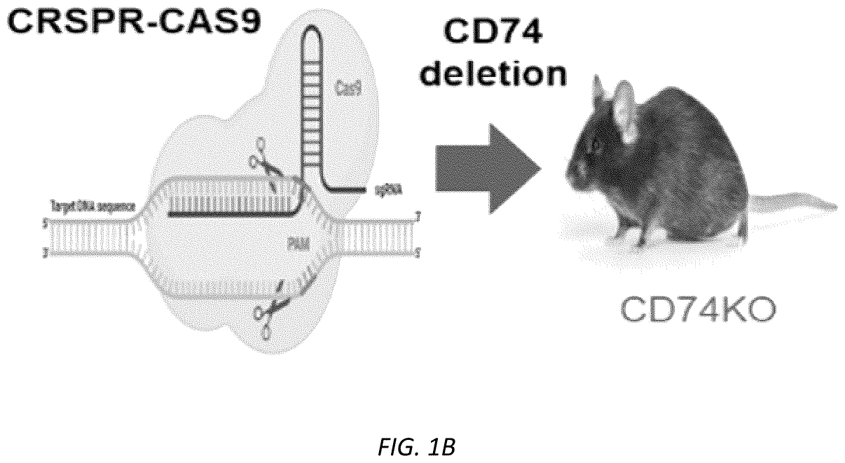

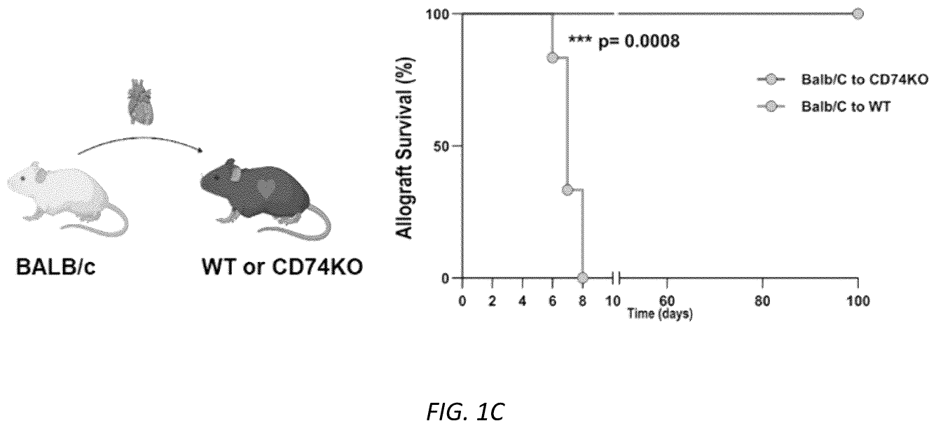

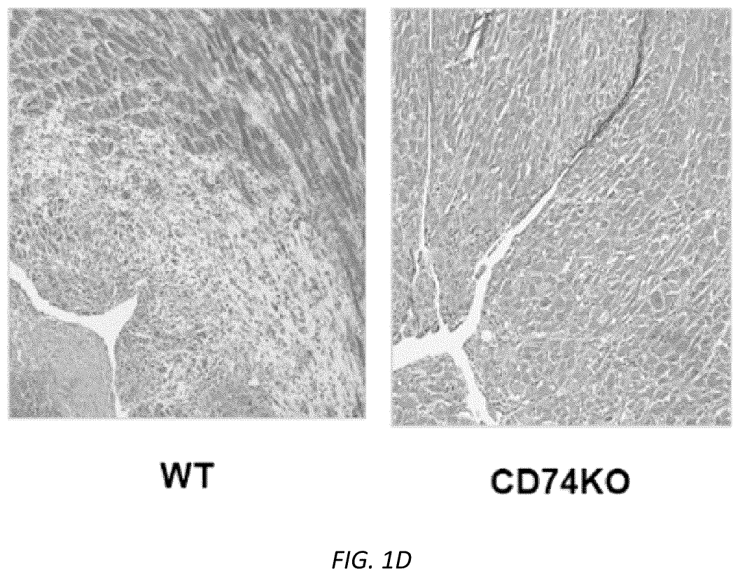

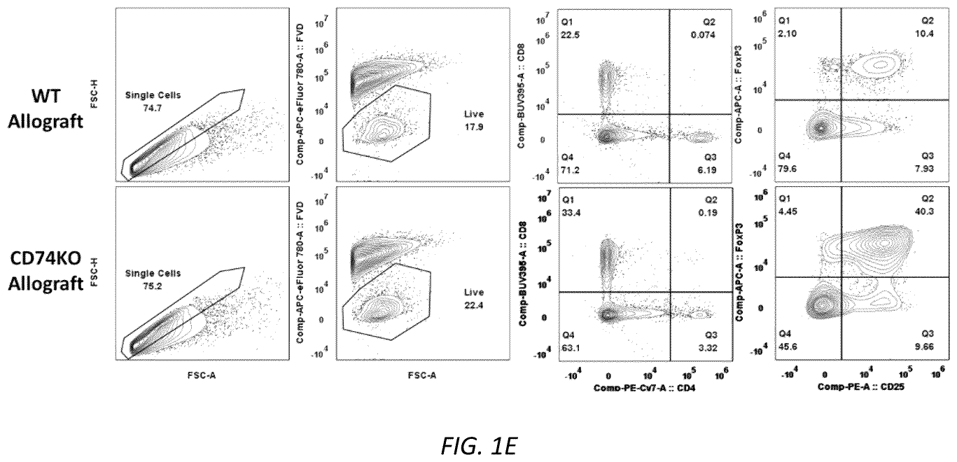

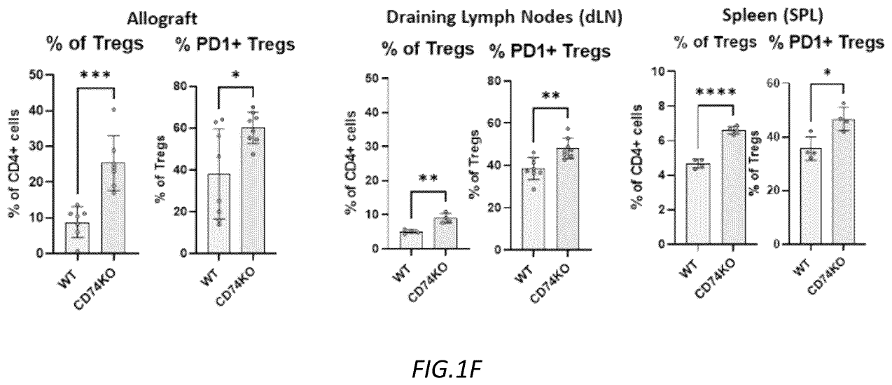

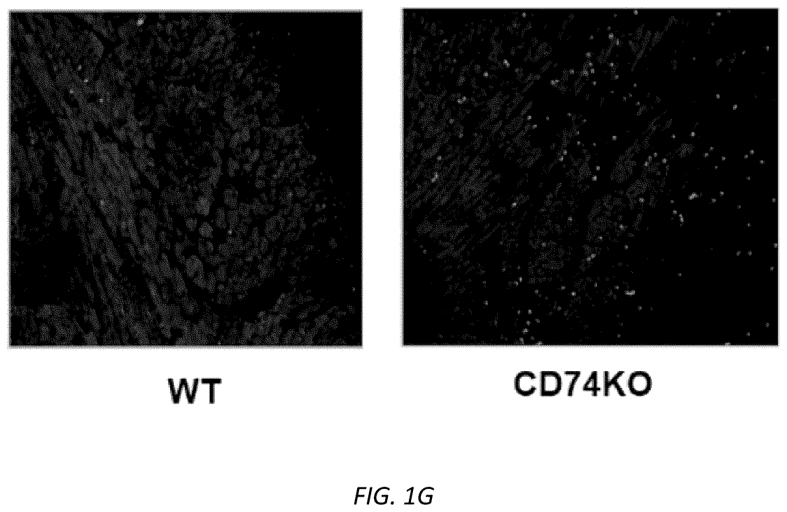

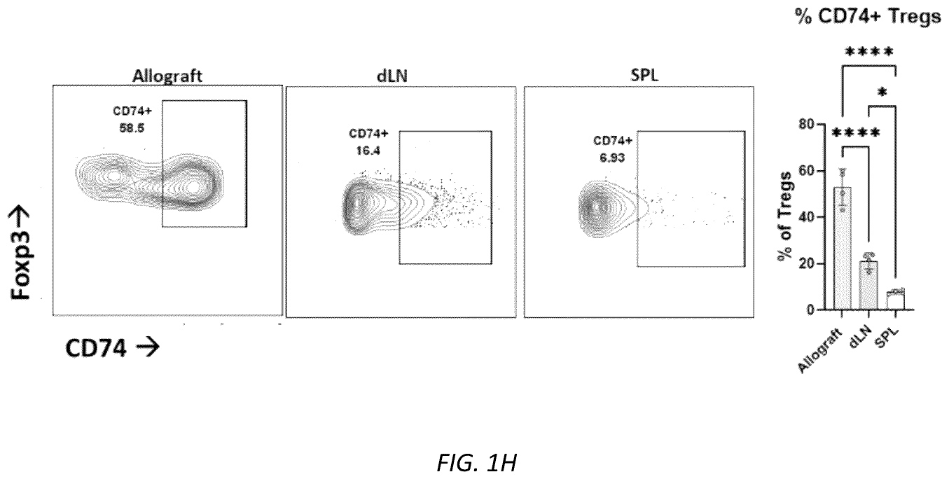

To study the effect of total CD74 deficiency on allograft survival, we performed heterotrophic cardiac transplant in a fully allogenic mismatch model, using BALB/c donors and either C57BL/6 (WT) or CD74KO recipients. Surprisingly, we observed indefinite cardiac allograft survival in CD74KO recipients (>100 days, p-value 0.008), as compared to a mean survival time (MST) of 7 days in WT recipients (FIG. 1C).

### Example 2. Heart Allografts of CD74KO Recipients Reveal Up to 3-Fold Increase in Infiltrating Treg Population with Pronounced Co-Inhibitory Marker Expression

To explore the mechanism behind this remarkable prolongation of allograft survival, WT and CD74KO mice recipients of BALB/c hearts were sacrificed on day 7 post-transplantation for mechanistic studies. Immunophenotyping of T cells harvested from spleen, graft draining lymph node (dLN) and heart allograft was performed using flow cytometry. Interestingly, we observed a 3-fold increase in the percentage of Tregs infiltrating the allografts of CD74KO recipients compared to WT (FIG. 1E). Increased frequency of Tregs was also evident in the spleen and dLN (FIG. 1F), but the difference was most prominent in the allograft and dLN. The PD-1/PDL-1 pathway is one of the known pathways through which Tregs induce cell death in their target effector cell. Our data showed a significant increase in PD-1⁺ Tregs in the heart allograft of CD74KO mice when compared to WT (FIG. 1F). Together, these findings suggest increased inhibitory potential of CD74KO Tregs. Moreover, histological analysis of allografts showed significantly less fibrosis and cellular infiltrate in the BALB/c allografts of CD74KO recipients (FIG. 1D). Additionally, Foxp3 staining revealed a higher Treg infiltrate in CD74KO allografts (FIG. 1G).

### Example 3. Stimulated WT Regulatory T Cells in the Allograft and dLN Show High CD74 Expression

To better understand the effect of CD74 on Tregs, we gauged the surface expression of CD74 by Tregs. We observed a significant increase in CD74 expression on WT Tregs infiltrating allograft and dLN, with a minimal expression on Tregs in the spleen (FIG. 1H).

### Example 4. Regulatory T Cells Play an Essential Role in the Maintenance of Allograft Survival in CD74KO Recipients

To explore the role of regulatory T cells (Tregs) in maintaining allograft tolerance exhibited in CD74 deficient mice, we performed cardiac allografts from BALB/c donor mice to CD74KO recipients. After confirmation of graft function on day 40 post-transplantation, one group of recipients were injected with 0.5 mg of anti-CD25 antibody intravenous on days 40 and 47 as previously shown by our group [18], to achieve Treg depletion. Treg depletion resulted in acute graft loss in all allografts with MST of 19 days after first injection, emphasizing the role of Tregs in allograft survival in CD74KO recipients (FIG. 1I).

### Example 5. MIF is Predominantly Expressed by CD4 and CD8 T Cells, Whereas CD74 is Predominantly Expressed by Tregs

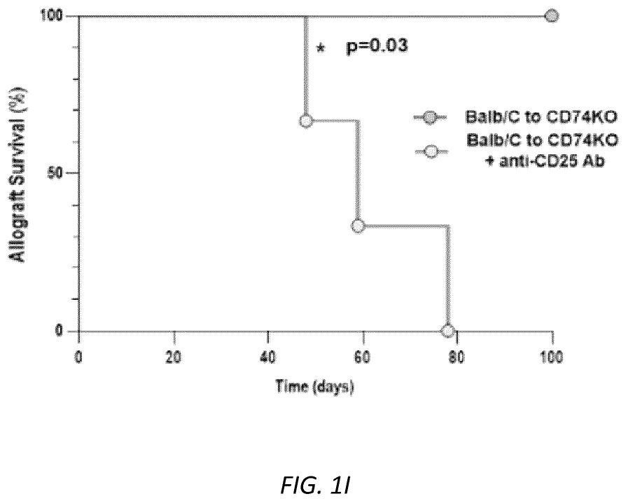

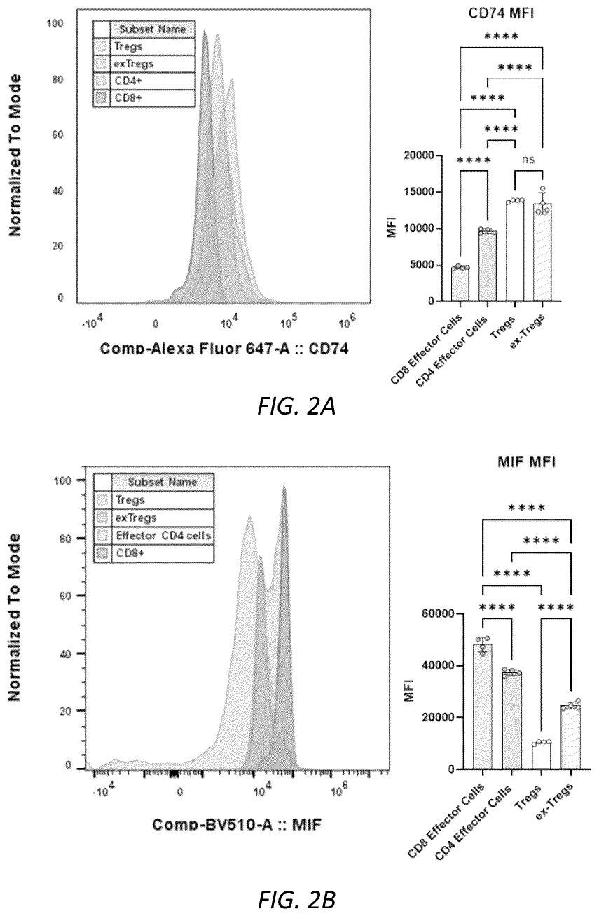

Tregs (CD4⁺Foxp3 GFP⁺ cells), CD8, and CD4 cells were isolated from the splenocytes of WT mice using Fluorescence-activated cell sorting (FACS). All cells were stimulated using plate-coated anti-CD3 (3 ug/ml) and anti-CD28 (3 ug/ml) for 48 hours. CD74 and MIF expression were assessed using flow cytometry. MIF was predominantly expressed by CD4 and CD8 effector cells, and by ex-Tregs (Tregs that lost their Foxp3 expression). However, compared to these latter cells, Tregs displayed a significantly lower MIF expression. On the contrary, CD74 expression was the highest on Tregs and ex-Tregs (FIGS. 2-B).

### Example 6. Regulatory T Cells Isolated from CD74KO Mice Display a Prominent Suppressive Function In-Vitro

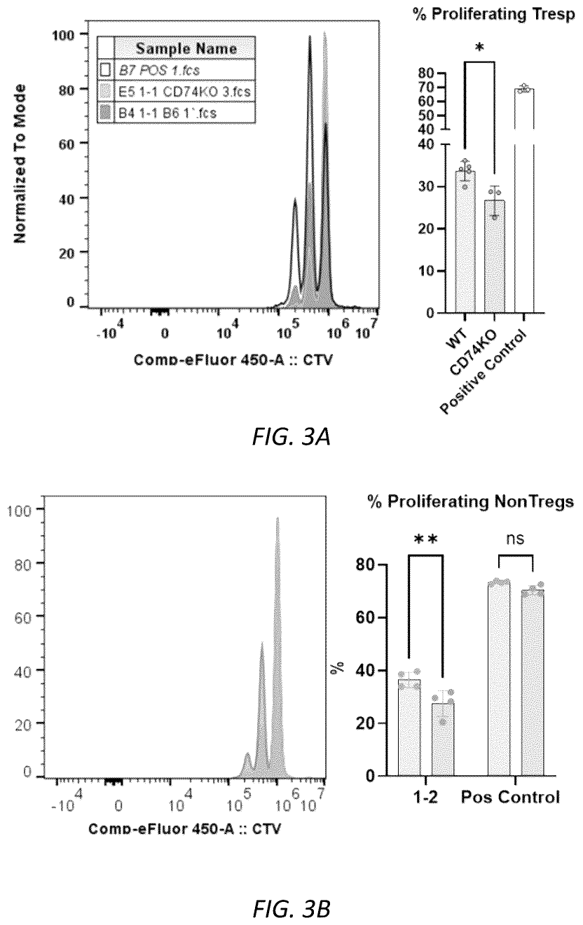

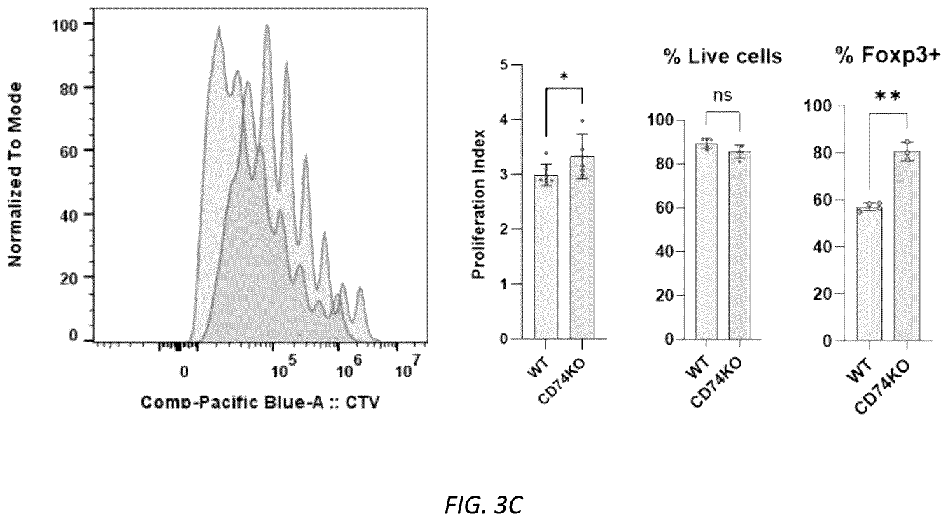

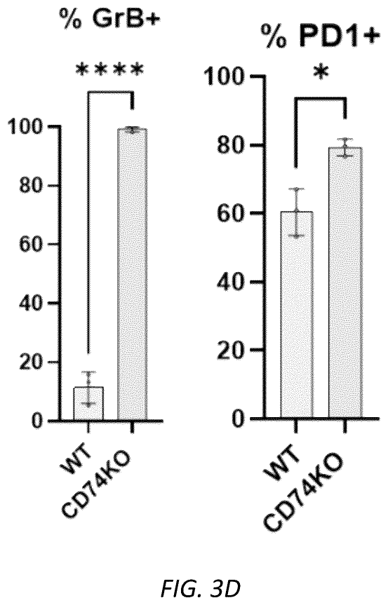

Magnetically activated cell sorting (MACS) isolated Tregs from WT and CD74KO mice were co-cultured with MACS-isolated activated WT CD4⁺CD25⁻ T cells (non-Tregs) at ratio of 1 to 2 respectively for 72 hours at 37° C. to evaluate the suppressive function of Tregs. Tregs from CD74KO mice induced significantly more reduction in proliferation of non-Tregs as compared to WT controls (FIG. 3A). Our group and others have previously shown Granzyme B (GrB) release, which activates caspase 3, as one of the ways Tregs induce cell death in their target cells [19, 20]. In-vitro stimulated CD74KO Tregs showed higher expression of Granzyme B and PD-1 as compared to WT counterparts (FIG. 3D). Together, these findings point towards increased suppressive function of CD74 deficient Tregs.

### Example 7. Blocking CD74 with a Monoclonal Antibody Potentiates the Suppressive Ability of WT Tregs

To upregulate CD74, we isolated Tregs from Foxp3-GFP WT mice and activated them in vitro with anti-CD3/anti-CD28 Dynabeads and IL-2 for 5 days. Then, to evaluate the suppressive function of the activated Tregs in the presence or absence of anti-CD74, GFP⁺ cells were sorted and co-cultured with MACS-isolated activated WT CD4⁺CD25⁻ T cells (non-Tregs) at a ratio of 1 to 2 for 72 hours at 37° C. Blocking CD74 on the Tregs with anti-CD74 induced a statistically significant reduction in non-Tregs proliferation (FIG. 3B). However, anti-CD74 had no effect on non-Tregs proliferation in the wells lacking Tregs (positive control).

### Example 8. CD74KO Tregs Exhibit Higher Proliferative Index In-Vitro and Maintained Higher Foxp3 Expression

To study the stability and survival characteristics of CD74KO Tregs, we isolated Tregs from CD74KO and WT mouse. They were then stained with cell trace violet (CTV) and stimulated in-vitro in presence of anti-CD3/anti-CD28 Dyna beads and IL-2 and cultured for 5 days. CD74KO Tregs exhibited significantly higher proliferation index compared to WT (FIG. 3C). After 5 days in culture, survival of Tregs, measured by frequency of cells negatively stained with viability dye, was comparable between groups (FIG. 3C). Meanwhile, significantly higher frequency of CD74KO Tregs stimulated in-vitro sustained FoxP3 expression compared to WT counterparts on day 5 (FIG. 3C), while both groups had similar purity of >95% on culture day 1. We hypothesize that this is due to higher Foxp3 stability in CD74KO.

### Example 9. Single-Cell RNA Sequencing Shows a Distinctive Phenotype for Allograft Infiltrating CD74 Deficient Tregs Compared to WT

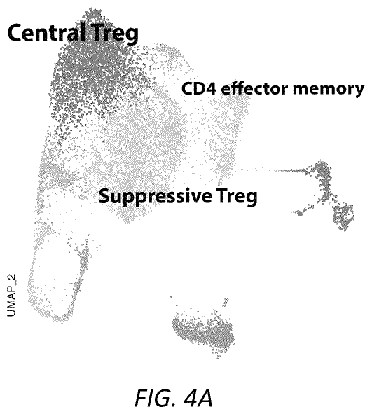

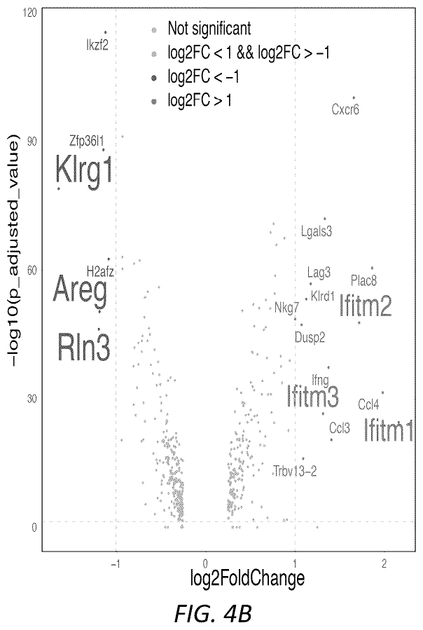

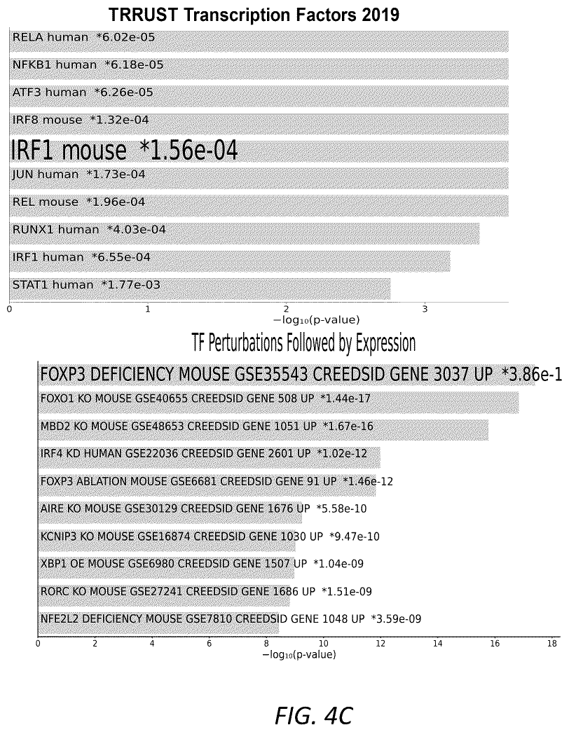

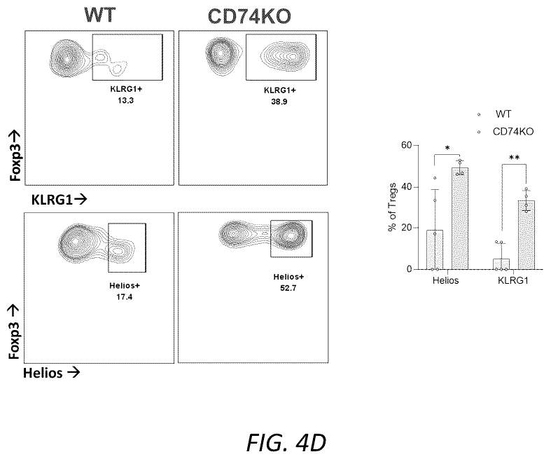

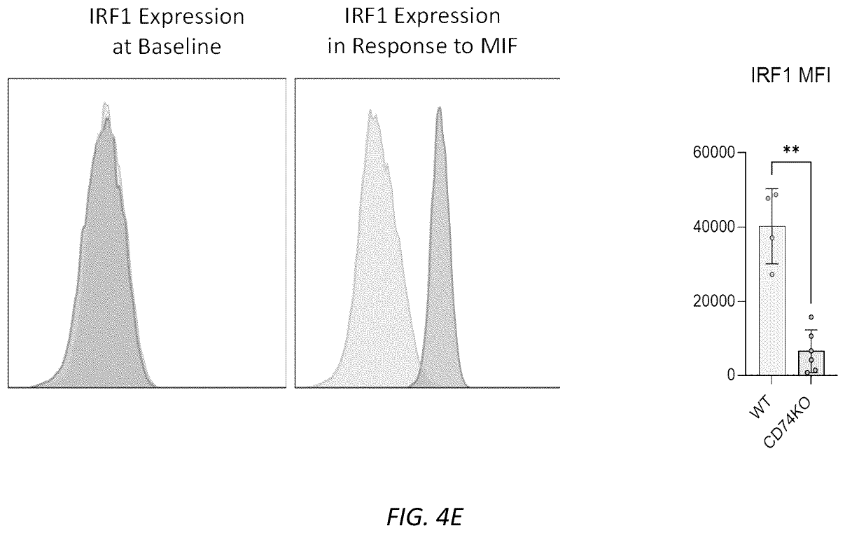

We performed single-cell RNA sequencing on CD4⁺CD25⁺ cells isolated from the allograft of CD74KO and WT recipients of BALB/c hearts. We identified two important Treg populations: central Tregs and suppressive Tregs (FIG. 4A). By comparing suppressive Tregs in WT vs. CD74KO mice, we found that CD74KO suppressive Tregs displayed significantly higher expression of genes involved in maintaining and mediating Tregs function: KLRG1, Areg, and kzf2 (helios gene) (FIG. 4B), whereas the WT suppressive Tregs upregulated ISGs such as Ifitm1, 2, and 3. Interestingly, a similar unbiased observation was described in murine models of lung adenocarcinoma. In these models, Tregs displayed an interferon-responsive phenotype with higher ISGs in less advanced tumors and a KLRG1⁺Areg⁺ effector phenotype in advanced tumors, suggesting that the ability of Tregs to suppress anti-tumor immunity in advanced tumors was mediated by the acquisition of the KLRG1⁺Areg⁺ phenotype [21]. Additionally, we used differentially expressed genes to study the transcription factor signatures of WT vs. CD74KO suppressive Tregs using TRRUST v2 database [22]. We found that IRF1 is downregulated in the CD74 deficient suppressive Treg and enriched in the wild type (FIG. 4C). Moreover, we studied the gene signature of WT suppressive Tregs using Enrichr [23], which showed that graft infiltrating suppressive Treg in the wild type had a similar gene signature as Foxp3 deficient mice in favor of a defective phenotype (FIG. 4C). Finally, we validated KLRG1 and Helios expression by flow cytometry in graft infiltrating Tregs (FIG. 4D).

### Example 10. MIF Activates IRF1 in WT but not in CD74KO Tregs

IRF1 has been shown to be a negative regulator of Tregs through direct repression of Foxp3 expression, leading to its instability in the setting of inflammation [24, 25]. Genetically deleting IRF1 in mice hindered autoimmunity [26-29]. It was then shown that IRF1^{-/-} mice display a higher percentage and absolute count of Tregs and that IRF1 deficient Tregs are more suppressive and stable than WT Tregs [24]. Given this, we validated our single-cell RNA seq data that suggested a downregulated IRF1 signaling in CD74KO Tregs at the protein level. We stimulated FACS sorted WT and CD74KO Tregs in vitro with anti-CD3/anti-CD28 Dynabeads and IL-2 for 5 days. Cells were rested overnight then stimulated with MIF (50 ng/ml) for 4 hours. IRF1 was significantly upregulated by MIF in WT Tregs but not in CD74KO Tregs (FIG. 4E). This finding endorses the superior suppressive phenotype of CD74KO Tregs.

### Example 11. CD74KO CD8 Effector Cells Produce Less Granzyme B and IFN-γ and are Less Alloreactive

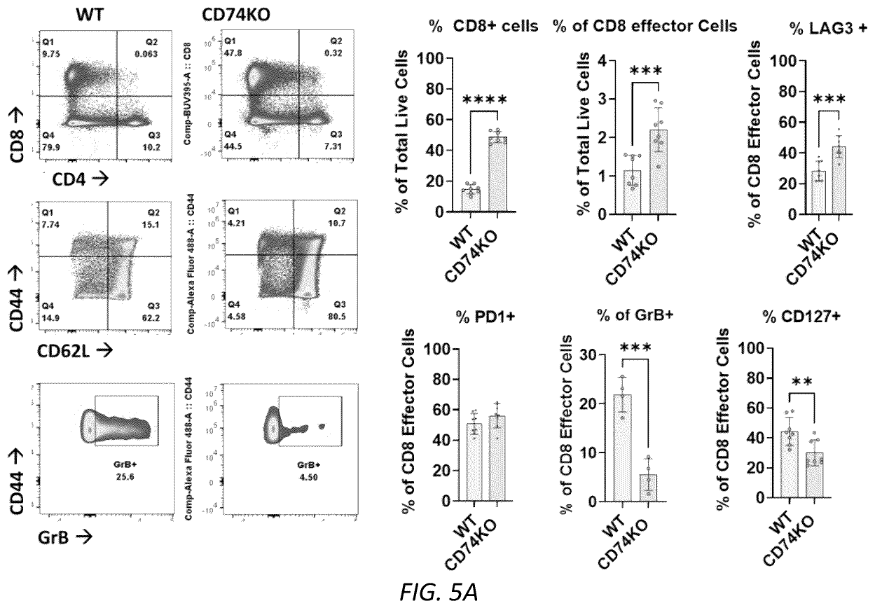

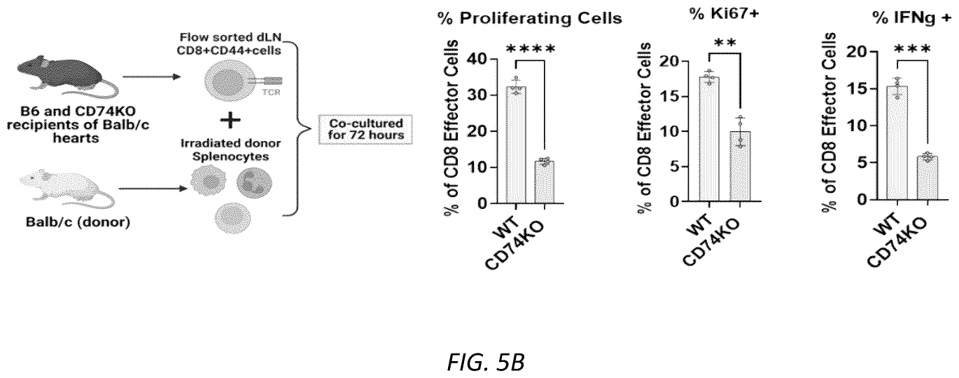

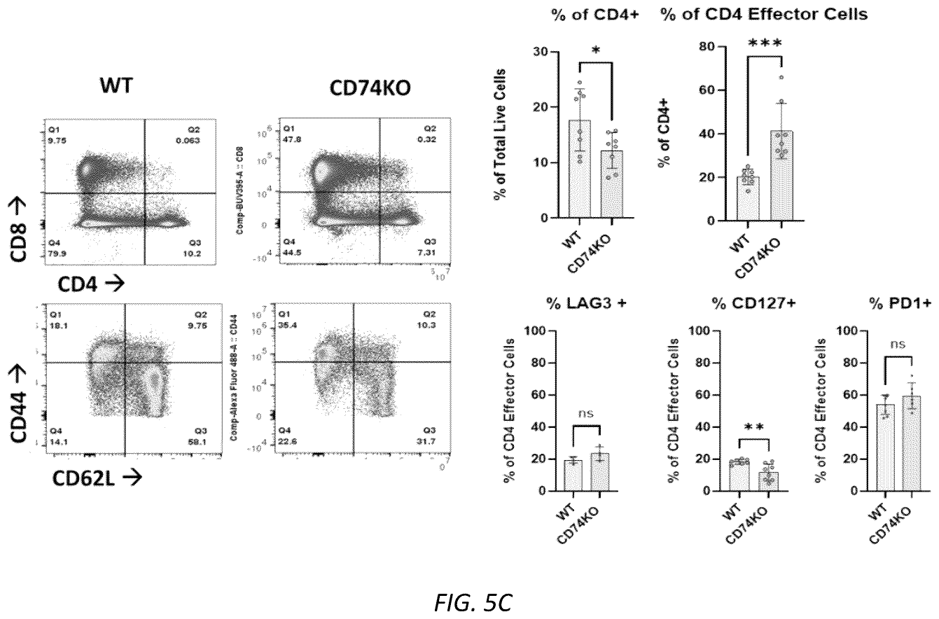

Flow cytometry analysis was performed on cell suspensions of dLNs of WT and CD74KO recipients of BALB/c hearts at day 7 post-transplantation. Although CD74KO mice exhibited no allograft rejection, the percentages of CD8 effector cells (CD8⁺CD44highCD62Llow) were significantly higher in the dLNs of CD74KO recipients when compared to WT recipients. However, CD8 effector cells expressed significantly higher levels of co-inhibitory molecules such as LAG3 with lower levels of functional markers such as CD127, which is often downregulated in exhausted cells (FIG. 5A). More importantly, these CD74 KO CD8 Teff released 4-fold less granzyme B compared to WT CD8 Teff (FIG. 5A). Furthermore, sorted CD8 effector cells from the dLNs of WT and CD74KO recipients of BALB/c hearts at day 7 post-transplantation were then stained with CTV and co-cultured with irradiated splenocytes of BALB/c mouse (donor) in a mixed lymphocytic reaction (MLR). CD74KO CD8 effector cells were significantly less reactive to donor antigen as evidenced by both % of proliferating Cells (tracked by CTV) and % of Ki67⁺ cells. They also released significantly less IFN-γ than the WT cells (FIG. 5B). A similar exhaustion phenotype was noted in CD4 Teff (FIG. 5C). These findings suggest that, in addition to promoting Tregs, CD74 deficiency also inhibits the pro-inflammatory activity and function of CD4 and CD8 effector cells.

### Example 12. CD74 Deletion Reduces the Proliferative Capacity of CD4 and CD8 T Cells In-Vitro and Impairs CD4 and CD8 T Cell Function In Vivo

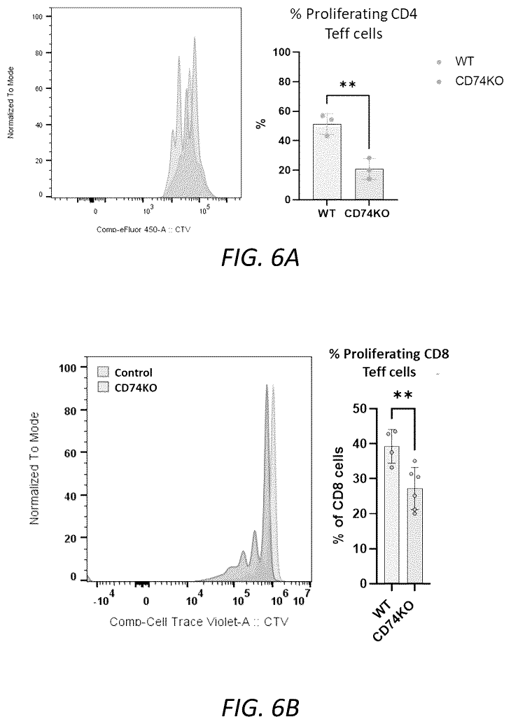

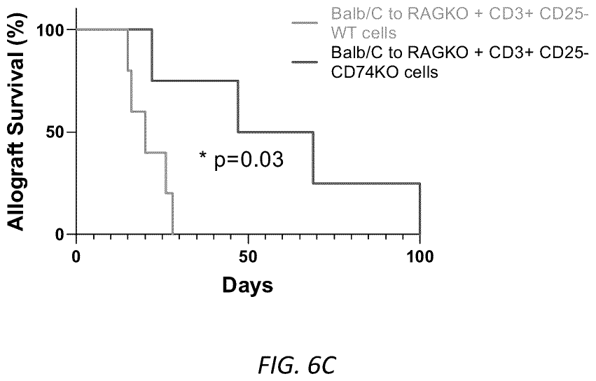

CD4⁺CD25⁻ and CD8 cells of WT and CD74KO mice were isolated and activated with plate-coated anti-CD3 and anti-CD28 for 72 hours. As shown in FIGS. 6A-B, both CD74KO CD4 (FIG. 6A) and CD8 T cells (FIG. 6B) proliferated significantly less than their corresponding WT T cells. Additionally, a significant prolongation in the BALB/c allografts survival of RAGKO recipients was achieved when CD74KO CD3⁺CD25⁻ cells mice were injected instead of WT CD3⁺CD25⁻ cells (FIG. 6C). Together, these findings highlight the negative effect of CD74 deletion on CD4⁺CD25⁻ and CD8 cells proliferation and pro-inflammatory activity both in vitro and in vivo.

### Example 13. Allograft-Infiltrating CD74KO Tregs are More Stable and Antigen-Specific than WT Tregs

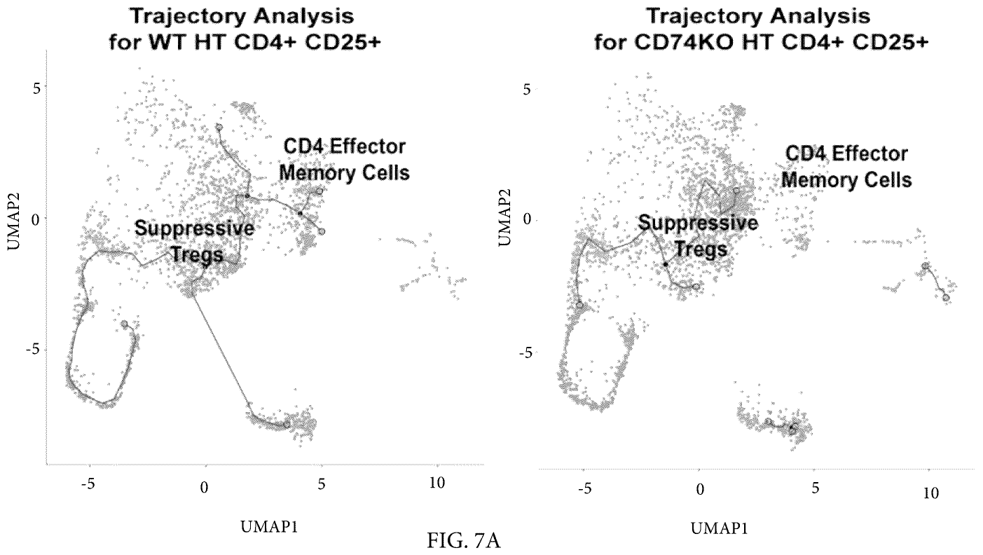

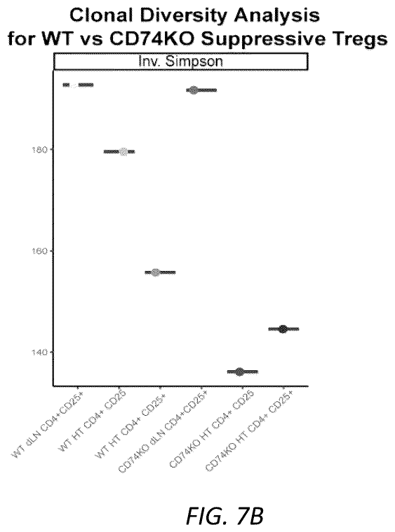

Trajectory analysis was performed to determine the fate of suppressive Tregs and their transition into effector CD4 cells. Interestingly, we observed a superior tendency for suppressive Tregs to transition into the CD4 effector memory cells cluster in WT, as evident by the trajectory followed by WT suppressive Tregs displaying spillover into CD4 effector memory cells cluster (FIG. 7A). Furthermore, T cell receptor (TCR) clonal analysis revealed interesting findings regarding the clonal diversity of allograft-infiltrating CD74KO Tregs compared to WT Tregs. Specifically, CD74KO Tregs displayed reduced clonal diversity, suggesting a tendency towards confinement within fewer T cell clones. This observation implies a potentially heightened antigen specificity among CD74KO Tregs when compared to their WT counterparts (FIG. 7B). Together, these findings support our hypothesis suggesting that CD74 deficient Tregs are more stable and suppressive than WT Tregs.

### Example 14. CD74KO Tregs are Less Responsive to IFNg than WT Tregs

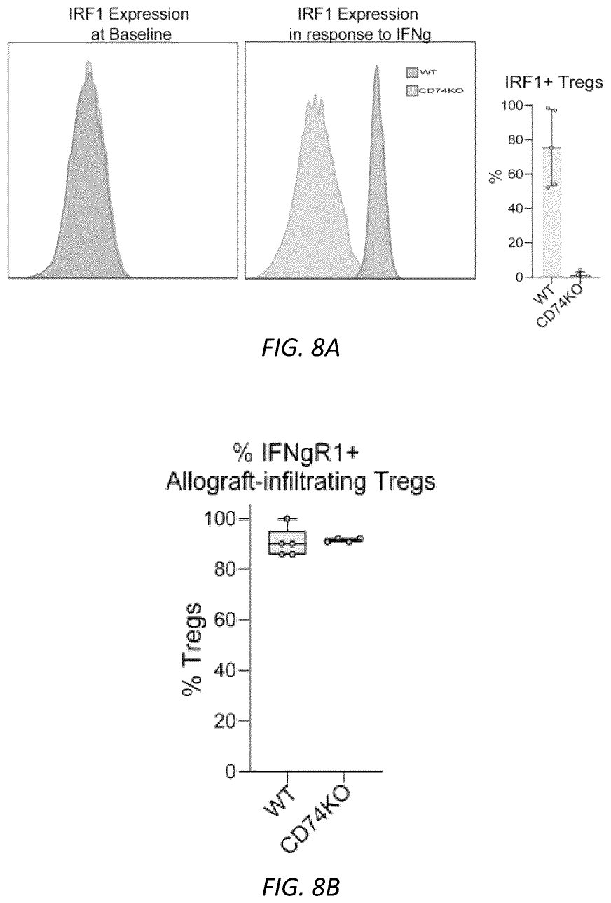

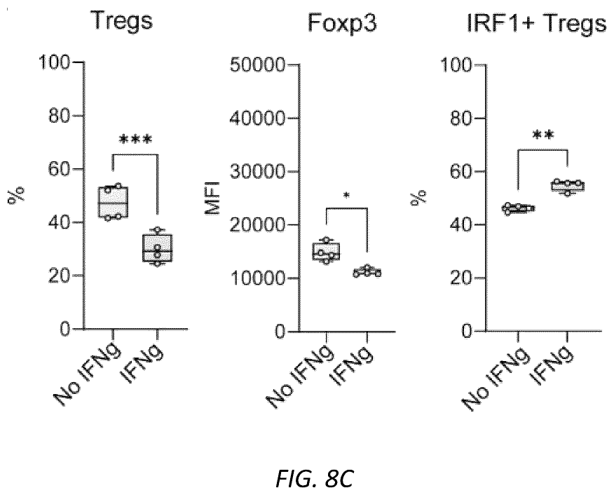

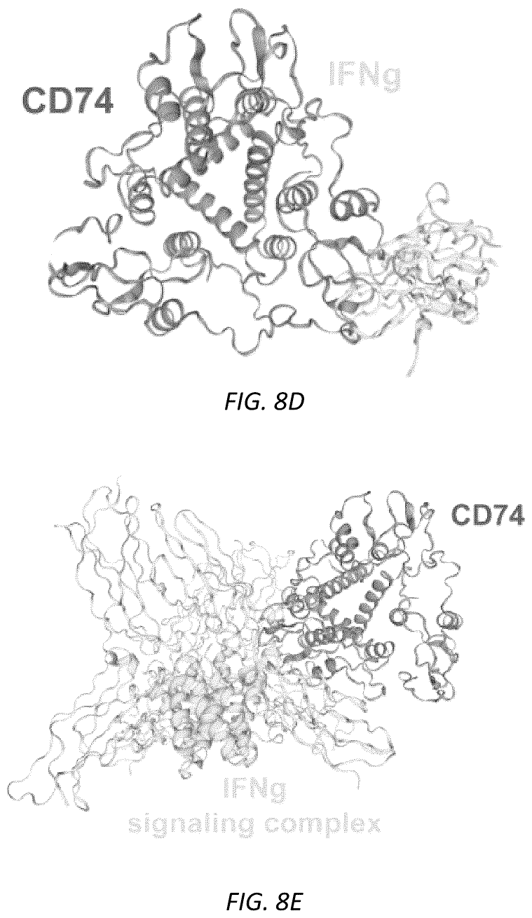

To validate our single cell RNA sequencing data, suggesting that CD74KO Tregs are less responsive to IFNg, at the protein level, we FACS-sorted WT and CD74KO Tregs and stimulated them in vitro with anti-CD3/anti-CD28 dyna beads and IL-2 for 5 days. Cells were rested overnight then stimulated with IFNg (1000 UI/ml) for 4 hours. IRF1 was significantly upregulated by IFNg in WT Tregs but not in CD74KO Tregs (FIG. 8A). Furthermore, we examined the level of IFNgR1 (a subunit of the IFNgR) expression by WT and CD74KO allograft-infiltrating Tregs. Surprisingly, CD74KO Tregs hypo-responsiveness to IFNg was not attributed to a lower expression of IFNg receptor (FIG. 8B). Collectively, these findings suggest that CD74 deficiency appears to interfere with the intracellular signaling cascade downstream of the IFNg receptor, rather than modulating the expression of the receptor itself. The diminished response to IFNg observed in CD74KO Tregs further underscores their heightened suppressive capacity as IFNg is known to affect Tregs stability as shown by us (FIG. 8C) and others [31].

Furthermore, to understand the interaction between IFNg and CD74, we conducted computational docking analysis to evaluate the potential interaction between CD74 and IFNg, as well as CD74 and IFNg signaling complex (IFNg plus IFNgR). The analysis revealed a pronounced tendency of CD74 to interact with IFNg as an isolated entity and also to interact with IFNg when bound to its receptor. Both interactions had a favorable docking score of less than-260 and a high confidence interval of more than 90% (FIG. 8D-E).

### Example 15. CD74fl/fl-UBC Cre Tregs are More Suppressive than WT Tregs

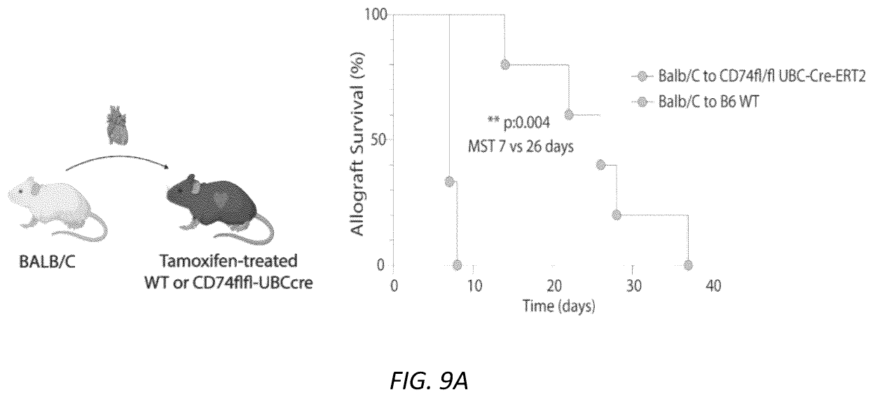

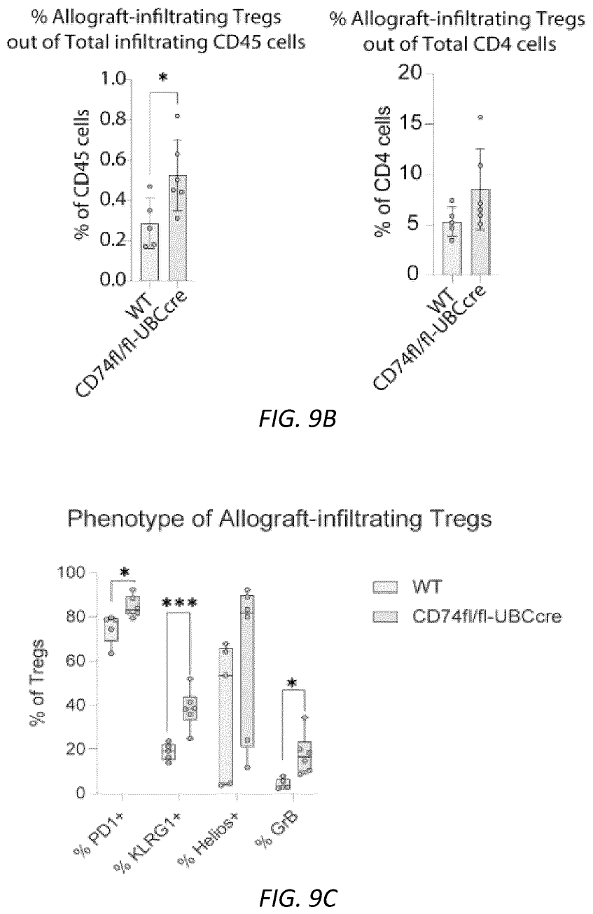

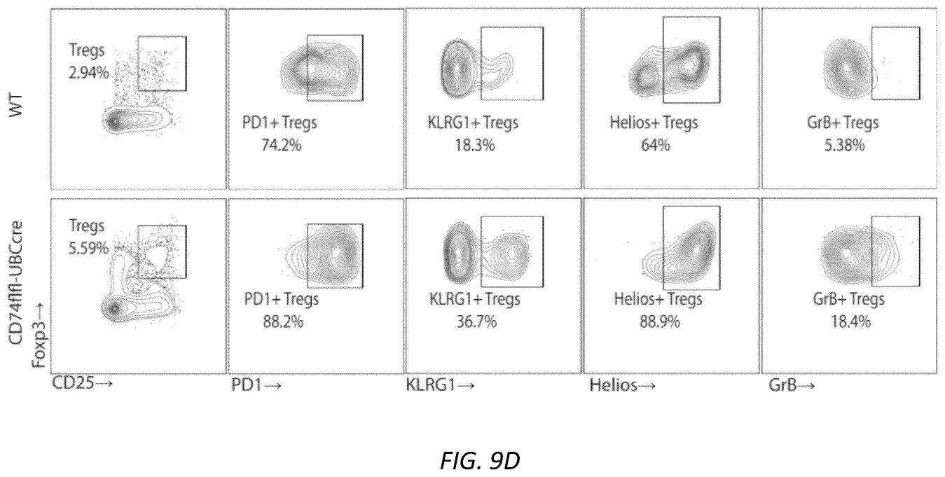

Thymic selection, occurring during embryonic and postnatal development stages, plays a crucial role in Tregs development and maturation. It ensures the generation of Tregs capable of suppressing and controlling autoimmune and alloimmune responses. Dysfunction in thymic Treg development can lead to autoimmune diseases, emphasizing the importance of the thymus in immune tolerance [32]. To investigate the impact of CD74 deficiency on thymic Treg selection, we established a conditional tamoxifen-inducible CD74 deficient model (CD74fl/fl-UBC cre) where CD74 deletion happens after thymic selection. Our data shows that CD74 deletion post-thymic selection using tamoxifen results in significant prolongation of allograft survival (FIG. 9A). This notable outcome is particularly impressive considering the temporal nature of tamoxifen-induced gene deletion. Moreover, at day 7 post-transplantation, allograft-infiltrating Tregs were significantly more abundant in the allograft of CD74fl/fl-UBC cre hosts (FIG. 9B). Similar to CD74KO Tregs, CD74fl/fl-UBC cre Tregs displayed a superior suppressive phenotype and had higher % of PD1, KLRG1 and GrB expression than WT Tregs (FIG. 9C-D).

### Example 16. CD74 Deletion in Tregs Using CRISPR-Cas Genome Editing Cas9 Lentivirus Generation and Testing

A one vector lentiviral CRISPR system was constructed by cloning pCAG-Cas9-2a-GFP and pU6-gRNA into the conventional lentiviral vector within the LTR. The CRISPR/Cas9 guides targeting CD74 genes were selected by feeding the full-length CD74 cDNA sequence into IDT's CRISPR guide designer tools. The top two ranked guide sequences were selected for testing experimentally. To generate lentivirus particles, this transfer plasmid was transfected into HEK293FT cells with two helper packaging plasmids. 48 hours after transfection, supernatant was concentrated by ultra-centrifugation at 30000 RPM for 2 hours and resuspended in ice-cold PBS. This freshly concentrated lentivirus was added on to HEK293T cells directly for transduction. 48 hours after transduction, the cells were checked for EGFP fluorescence to monitor transduction efficiency.

gRNA Spacer Sequence:

| | CD74_G1: |
|---|---|
| (SEQ ID NO: 1) | |
| | GCATGAACAGTTGCCCATACT |
| | CD74_G2: |
| (SEQ ID NO: 2) | |
| | GAGGGAGTAGCCATCCGCATC |

### Cas9 Protein Sequence Used:

(SEQ ID NO: 3)
MDYKDHDGDYKDHDIDYKDDDDKMAPKKKRKVGIHGVPAADKKYSIGLDI
GTNSVGWAVITDEYKVPSKKFKVLGNTDRHSIKKNLIGALLFDSGETAEA
TRLKRTARRRYTRRKNRICYLQEIFSNEMAKVDDSFFHRLEESFLVEEDK
KHERHPIFGNIVDEVAYHEKYPTIYHLRKKLVDSTDKADLRLIYLALAHM
IKFRGHFLIEGDLNPDNSDVDKLFIQLVQTYNQLFEENPINASGVDAKAI
LSARLSKSRRLENLIAQLPGEKKNGLFGNLIALSLGLTPNFKSNFDLAED
AKLQLSKDTYDDDLDNLLAQIGDQYADLFLAAKNLSDAILLSDILRVNTE
ITKAPLSASMIKRYDEHHQDLTLLKALVRQQLPEKYKEIFFDQSKNGYAG
YIDGGASQEEFYKFIKPILEKMDGTEELLVKLNREDLLRKQRTFDNGSIP
HQIHLGELHAILRRQEDFYPFLKDNREKIEKILTFRIPYYVGPLARGNSR
FAWMTRKSEETITPWNFEEVVDKGASAQSFIERMTNFDKNLPNEKVLPKH
SLLYEYFTVYNELTKVKYVTEGMRKPAFLSGEQKKAIVDLLFKTNRKVTV
KQLKEDYFKKIECFDSVEISGVEDRFNASLGTYHDLLKIIKDKDFLDNEE
NEDILEDIVLTLTLFEDREMIEERLKTYAHLFDDKVMKQLKRRRYTGWGR
LSRKLINGIRDKQSGKTILDFLKSDGFANRNFMQLIHDDSLTFKEDIQKA
QVSGQGDSLHEHIANLAGSPAIKKGILQTVKVVDELVKVMGRHKPENIVI
EMARENQTTQKGQKNSRERMKRIEEGIKELGSQILKEHPVENTQLQNEKL
YLYYLQNGRDMYVDQELDINRLSDYDVDHIVPQSFLKDDSIDNKVLTRSD
KNRGKSDNVPSEEVVKKMKNYWRQLLNAKLITQRKFDNLTKAERGGLSEL
DKAGFIKRQLVETRQITKHVAQILDSRMNTKYDENDKLIREVKVITLKSK
LVSDFRKDFQFYKVREINNYHHAHDAYLNAVVGTALIKKYPKLESEFVYG
DYKVYDVRKMIAKSEQEIGKATAKYFFYSNIMNFFKTEITLANGEIRKRP
LIETNGETGEIVWDKGRDFATVRKVLSMPQVNIVKKTEVQTGGFSKESIL
PKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRK
RMLASAGELQKGNELALPSKYVNFLYLASHYEKLKGSPEDNEQKQLFVEQ
HKHYLDEIIEQISEFSKRVILADANLDKVLSAYNKHRDKPIREQAENIIH
LFTLTNLGAPAAFKYFDTTIDRKRYTSTKEVLDATLIHQSITGLYETRID
LSQLGGDKRPAATKKAGQAKKKKEFGSGEGRGSLLTCGDVEENPGPVSKG
EELFTGVVPILVELDGDVNGHKFSVSGEGEGDATYGKLTLKFICTTGKLP
VPWPTLVTTLTYGVQCFSRYPDHMKQHDFFKSAMPEGYVQERTIFFKDDG
NYKTRAEVKFEGDTLVNRIELKGIDFKEDGNILGHKLEYNYNSHNVYIMA
DKQKNGIKVNFKIRHNIEDGSVQLADHYQQNTPIGDGPVLLPDNHYLSTQ
SALSKDPNEKRDHMVLLEFVTAAGITLGMDELYKEF

Human Tregs Transfection with CD74-Cas9

CD74 can be deleted in the human Tregs of subjects undergoing organ transplantation using the CRISPR/Cas process. Tregs are isolated from the peripheral blood mononuclear cells of the subjects using the human CD4+CD25+ CD127dim/-Regulatory T Cell Isolation Kit II (130-094-775, Miltenyi Biotecs). One day before nucleofection, Tregs are stimulated in a 6-well plate at a density of 250.000 cells/mL in 2 mL X-vivo+5% FBS and 100 U/mL IL-2 (11147528001, Sigma-Aldrich) or 500 IU/mL Proleukin® (Novartis). For transfection, cells are collected, centrifuged at 90 g for 10 minutes at room temperature, and 1 million Tregs are resuspended in 20 μl P3 Primary Cell 4D-Nucleofector X Kit S (V4XP-3032, Lonza) [6]. In PCR tubes, 20 pmol Cas9 nuclease (9212-0.25 MG, Aldevron) is mixed with 100 pmol CD74 sgRNA and incubated at 37° C. for a minimum of 10 minutes before adding to the cells. The cell/RNP mixture is then transferred to Nucleofection cuvette strips (4D-Nucleofector X Kit S, Lonza) and cells are electroporated using the 4D-Nucleofector Core Unit (AAF-1002B, Lonza) and X Unit (AAF-1002X, Lonza) with program EO115 [6]. After transfection, 80 μl medium at room temperature (X-vivo+5% FBS+100 U/mL IL-2 or 500 IU/mL Proleukin®) is added to the wells of the cuvette strip. Cells are then collected and plated in 1 mL pre-warmed medium in 24-well plates and incubated at 37° C. until read-out [6].

### REFERENCES

1. Braza, F., et al., Role of TLRs and DAMPs in allograft inflammation and transplant outcomes. Nat Rev Nephrol, 2016. 12 (5): p. 281-90.
2. Ravindranath, M. H., F. El Hilali, and E. J. Filippone, The Impact of Inflammation on the Immune Responses to Transplantation: Tolerance or Rejection? Front Immunol, 2021. 12: p. 667834.
3. Gao, Y., et al., Inflammation negatively regulates FOXP3 and regulatory T-cell function via DBC1. Proceedings of the National Academy of Sciences, 2015. 112 (25): p. E3246-E3254.
4. El Fekih, R., et al., Discovery and Validation of a Urinary Exosome mRNA Signature for the Diagnosis of Human Kidney Transplant Rejection. J Am Soc Nephrol, 2021. 32 (4): p. 994-1004.
5. Van Zeebroeck, L., et al., Fast and Efficient Genome Editing of Human FOXP3+ Regulatory T Cells. Frontiers in Immunology, 2021. 12:655122.
6. Calandra, T. and T. Roger, Macrophage migration inhibitory factor: a regulator of innate immunity. Nat Rev Immunol, 2003. 3 (10): p. 791-800.
7. Tilstam, P. V., et al., MIF but not MIF-2 recruits inflammatory macrophages in an experimental polymicrobial sepsis model. J Clin Invest, 2021. 131 (23) e127171.
8. Calandra, T., et al., Protection from septic shock by neutralization of macrophage migration inhibitory factor. Nat Med, 2000. 6 (2): p. 164-70.
9. Bozza, M., et al., Targeted Disruption of Migration Inhibitory Factor Gene Reveals Its Critical Role in Sepsis. Journal of Experimental Medicine, 1999. 189 (2): p. 341-346.
10. Schröder, B., The multifaceted roles of the invariant chain CD74-More than just a chaperone. Biochimica et Biophysica Acta (BBA)-Molecular Cell Research, 2016. 1863 (6, Part A): p. 1269-1281.
11. Su, H., et al., The biological function and significance of CD74 in immune diseases. Inflamm Res, 2017. 66 (3): p. 209-216.
12. Shi, X., et al., CD44 is the signaling component of the macrophage migration inhibitory factor-CD74 receptor complex. Immunity, 2006. 25 (4): p. 595-606.
13. Leng, L., et al., MIF signal transduction initiated by binding to CD74. J Exp Med, 2003. 197 (11): p. 1467-76.
14. Nakamura, A., et al., Macrophage migration inhibitory factor drives pathology in a mouse model of spondyloarthritis and is associated with human disease. Sci Transl Med, 2021. 13 (616): p. eabg1210.
15. David, K., et al., CD74 as a regulator of transcription in normal B cells. Cell Rep, 2022. 41 (5): p. 111572.
16. Yamada, A., et al., Cutting Edge: Recipient MHC Class II Expression Is Required to Achieve Long-Term Survival of Murine Cardiac Allografts After Costimulatory Blockade1. The Journal of Immunology, 2001. 167 (10): p. 5522-5526.
17. Pietra, B. A., et al., CD4 T cell-mediated cardiac allograft rejection requires donor but not host MHC class II. J Clin Invest, 2000. 106 (8): p. 1003-10.
18. Kawano, Y., et al., Blocking IFNAR1 inhibits multiple myeloma-driven Treg expansion and immunosuppression. J Clin Invest, 2018. 128 (6): p. 2487-2499.
19. Azzi, J., et al., Serine protease inhibitor 6 plays a critical role in protecting murine granzyme B-producing regulatory T cells. J Immunol, 2013. 191 (5): p. 2319-27.
20. Cao, X., et al., Granzyme B and perforin are important for regulatory T cell-mediated suppression of tumor clearance. Immunity, 2007. 27 (4): p. 635-46.
21. Li, A., et al., IL-33 Signaling Alters Regulatory T Cell Diversity in Support of Tumor Development. Cell Rep, 2019. 29 (10): p. 2998-3008.e8.
22. Han, H., et al., TRRUST v2: an expanded reference database of human and mouse transcriptional regulatory interactions. Nucleic Acids Research, 2017. 46 (D1): p. D380-D386.
23. Chen, E. Y., et al., Enrichr: interactive and collaborative HTML5 gene list enrichment analysis tool. BMC Bioinformatics, 2013. 14 (1): p. 128.
24. Fragale, A., et al., IFN regulatory factor-1 negatively regulates CD4+CD25+ regulatory T cell differentiation by repressing Foxp3 expression. J Immunol, 2008. 181 (3): p. 1673-82.
25. Lal, G., et al., Distinct inflammatory signals have physiologically divergent effects on epigenetic regulation of Foxp3 expression and Treg function. Am J Transplant, 2011. 11 (2): p. 203-14.
26. Tada, Y., et al., Reduced incidence and severity of antigen-induced autoimmune diseases in mice lacking interferon regulatory factor-1. J Exp Med, 1997. 185 (2): p. 231-8.
27. Buch, T., C. Uthoff-Hachenberg, and A. Waisman, Protection from autoimmune brain inflammation in mice lacking IFN-regulatory factor-1 is associated with Th2-type cytokines. Int Immunol, 2003. 15 (7): p. 855-9.
28. Sommer, F., et al., Lack of gastritis and of an adaptive immune response in interferon regulatory factor-1-deficient mice infected with Helicobacter pylori. Eur J Immunol, 2001. 31 (2): p. 396-402.
29. Nakazawa, T., et al., Complete suppression of insulitis and diabetes in NOD mice lacking interferon regulatory factor-1. J Autoimmun, 2001. 17 (2): p. 119-25.

30 Uehara, M., et al., Regulation of T cell alloimmunity by PI3Kgamma and PI3Kdelta. Nat Commun, 2017. 8 (1): p. 951.
31 Overacre-Delgoffe, A. E., et al., Interferon-γ Drives T (reg) Fragility to Promote Anti-tumor Immunity. Cell, 2017. 169 (6): p. 1130-1141.e11.
32 Hsieh, C.-S., H.-M. Lee, and C.-W. J. Lio, Selection of regulatory T cells in the thymus. Nature Reviews Immunology, 2012. 12 (3): p. 157-167.

### OTHER EMBODIMENTS

Whilst the invention has been disclosed in particular embodiments, it will be understood by those skilled in the art that certain substitutions, alterations and/or omissions may be made to the embodiments without departing from the spirit of the invention. Accordingly, the foregoing description is meant to be exemplary only, and should not limit the scope of the invention. All references, scientific articles, patent publications, and any other documents cited herein are hereby incorporated by reference for the substance of their disclosure.

We claim:

1. A method of promoting transplantation tolerance in a subject, the method comprising administering a therapeutically effective amount of an anti-CD-74 antibody to the subject.

2. The method according to claim 1, wherein the anti-CD74 antibody is administered prior to, simultaneously to, or subsequent to, transplantation of an organ into the subject.

3. The method according to claim 1, wherein the anti-CD74 antibody is Milatuzumab (Invitrogen MA5-41757).

4. The method according to claim 1, wherein the subject is a human subject.

5. A method of promoting organ survival in in an organ transplanted into a subject, the method comprising administering a therapeutically effective amount of an anti-CD-74 antibody to the subject.

6. The method according to claim 5, wherein the anti-CD74 antibody is administered prior to, simultaneously to, or subsequent to, transplantation of an organ into the subject.

7. The method according to claim 5, wherein the anti-CD74 antibody is Milatuzumab (Invitrogen MA5-41757).

8. The method according to claim 5, wherein the subject is a human subject.

9. A method of preparing a population of CD74-deficient T reg cells, the method comprising:
providing a population of CD4+Foxp3+ Treg cells, or precursors thereof; and
using a Cas enzyme to edit genomic DNA of the cells to introduce a mutation that knocks out CD74;
thereby preparing a population of CD74-deficient T reg cells.

10. The method according to claim 9, wherein the precursors are CD34+ hematopoietic stem cells (HSCs) or induced pluripotent stem cells (iPSCs), and the methods further comprise maturing the precursors in vitro to produce Treg cells.

11. The method according to claim 9, wherein the Cas enzyme is a nuclease or nickase, or is in a base editor or prime editor.

12. The method according to claim 9, wherein the Cas enzyme is Streptococcus pyogenes Cas9 (SpCas9) and the gRNAs comprise spacer sequences

| (SEQ ID NO: 1) | |
|---|---|
| | GCATGAACAGTTGCCCATACT |
| | and/or |
| (SEQ ID NO: 2) | |
| | GAGGGAGTAGCCATCCGCATC |

13. The method according to claim 9, wherein the cells are from a human subject.

14. A composition comprising CD74 deficient Treg cells, prepared by the method of claim 9.

15. The composition according to claim 14, wherein the CD74-deficient Treg cells exhibit more immunosuppression as compared to wild type Treg cells.

16. The composition according to claim 15, wherein the CD74 deficient Treg cells release higher levels of suppressive cytokines as compared to wild type Treg cells.

17. The composition according to claim 15, wherein the CD74 deficient Treg cells express higher levels of co-inhibitory molecules as compared to wild type Treg cells.

18. A method of promoting transplantation tolerance in a subject, or promoting organ survival in an organ transplanted into a subject, the method comprising administering a therapeutically effective amount of the composition comprising CD74 deficient Treg cells of claim 14 to the subject.

19. The method according to claim 18, wherein the CD74 deficient Treg cells are administered prior to, simultaneously to, or subsequent to, transplantation of an organ into the subject.

20. The method according to claim 18, wherein the subject is a human subject.

21. The method of claim 18, wherein the CD74 deficient Treg cells are prepared using CD4+Foxp3+ Treg cells, or precursors thereof that are autologous to the subject.
